(19) United States
(12) Patent Application Publication
(10) Pub. No.: US 2015/0057774 A1
(43) Pub. Date: Feb. 26, 2015

(54) GRAPHICAL USER INTERFACE FOR SURGICAL CONSOLE

(71) Applicant: NOVARTIS AG, BASEL (CH)

(72) Inventors: MIKHAIL BOUKHNY, LAGUNA NIGUEL, CA (US); KEVIN KING, LAGUNA HILLS, CA (US); TIFFANY SUTLIFF, FULLERTON, CA (US); DAVID A. THOE, LAGUNA HILLS, CA (US); CRAIG WOOLDRIDGE, IRVINE, CA (US); MARK YOUNG, LAGUNA NIGUEL, CA (US)

(21) Appl. No.: 14/333,823

(22) Filed: Jul. 17, 2014

Related U.S. Application Data
(60) Provisional application No. 61/868,672, filed on Aug. 22, 2013.

(51) Int. Cl. A61B 19/00; G05B 15/02

(52) U.S. Cl. CPC A61B 19/56; G05B 15/02
USPC 700/90

(57) ABSTRACT

A surgical system includes a control device, a touch screen display, and a GUI displayed on the touch screen display. A parameter icon has a parameter area, a first set point, a second set point, a parameter line extending from the first set point to the second set point, and a value orb located on the parameter line. The location of the value orb on the parameter line corresponds to a control position of the control device. A parameter value area is located under the parameter line and extends from the first set point to the value orb. A numerical parameter value is located in the parameter area. The parameter line represents a range of parameter values from the first set point to the second set point, and when the control device is a foot pedal, a range of travel of the foot pedal.

### BACKGROUND

The present invention relates to surgical systems and more particularly to a graphical user interface (GUI) for a surgical console.

The human eye can suffer a number of maladies causing mild deterioration to complete loss of vision. While contact lenses and eyeglasses can compensate for some ailments, ophthalmic surgery is required for others. Generally, ophthalmic surgery is classified into posterior segment procedures, such as vitreoretinal surgery, and anterior segment procedures, such as cataract surgery.

The surgical instrumentation used for ophthalmic surgery can be specialized for anterior segment procedures or posterior segment procedures or support both. Such surgical instrumentation can comprise a vitreoretinal and cataract microsurgical console. Such a surgical console can provide a variety of functions depending on the surgical procedure and surgical instrumentation. For example, surgical consoles can expedite cataract surgeries (e.g. phacoemulsification procedures) by helping manage irrigation and aspiration flows into and out of a surgical site. And of course surgical consoles can provide other functions.

Thus, vitreoretinal and cataract surgical consoles usually have a large set of functionality such as cutting, vacuum, etc. and commensurately are amenable to a large degree of customization. In other words, each of the parameters of such a surgical console may be individually adjusted to achieve desired settings. The adjustment of these parameters may need to be coordinated (e.g. the setting of one parameter depends at least in part on the settings of one or more other parameters) for best performance or to avoid possible injury or complications. This requirement may mean that settings corresponding to multiple parameters may need to be verified, calculated or adjusted even if a doctor is concerned only with a single parameter. Not only do these adjustments consume more time, but in addition, they may increase the chances of mistakes being made in the configuration of the surgical console, which, in some instances, may lead to injury of a patient or a doctor performing a surgical procedure. Finally, the presentation of settings and parameters on the console is also important so that the doctor can clearly see the status of the console and the procedure.

Therefore there is a need for a system or method for configuring a surgical console and presenting parametric data during operation of the console.

### SUMMARY OF THE INVENTION

The present disclosure describes several examples of the invention. In one example, a surgical system includes a control device, a touch screen display, and a GUI displayed on the touch screen display. A parameter icon has a parameter area, a first set point, a second set point, a parameter line extending from the first set point to the second set point, and a value orb located on the parameter line. The location of the value orb on the parameter line corresponds to a control position of the control device. A parameter value area is located under the parameter line and extends from the first set point to the value orb. A numerical parameter value is located in the parameter area. The parameter line represents a range of parameter values from the first set point to the second set point, and when the control device is a foot pedal, a range of travel of the foot pedal.

In another example, a surgical system includes a control device, a touch screen display, and a GUI displayed on the touch screen display. A parameter icon has a parameter area, a first set point, a second set point, a third set point, a mid-line, a first parameter line extending from the first set point to the mid-line, a second parameter line extending from the mid-line to the third set point, and a value orb located on the first or second parameter line. The location of the value orb on the first or second parameter line corresponds to a control position of the control device. A parameter value area is located under the first parameter line and extends from the first set point to the value orb. A numerical parameter value is located in the parameter area. The first parameter line represents a first range of travel of the foot pedal, and the second parameter line represents a second range of travel of the foot pedal. The first parameter line represents a first range of parameter values from the first set point to the second set point, and the second parameter line represents a second range of parameter values from the second set point to the third set point. The GUI may also comprise a fourth set point, a transition line, and a third parameter line extending from the transition line to the fourth set point.

In another example, a computer readable medium has a set of computer instructions for presenting a GUI with a parameter icon. The parameter icon has a parameter area, a first set point, a second set point, a parameter line extending from the first set point to the second set point, and a value orb located on the parameter line. The location of the value orb on the parameter line corresponds to a control position of the control device. A parameter value area is located under the parameter line and extends from the first set point to the value orb. A numerical parameter value is located in the parameter area. The parameter line represents a range of parameter values from the first set point to the second set point, and when the control device is a foot pedal, a range of travel of the foot pedal.

In another example, a computer readable medium has a set of computer instructions for presenting a GUI with a parameter icon. The parameter icon has a parameter area, a first set point, a second set point, a third set point, a mid-line, a first parameter line extending from the first set point to the mid-line, a second parameter line extending from the mid-line to the third set point, and a value orb located on the first or second parameter line. The location of the value orb on the first or second parameter line corresponds to a control position of the control device. A parameter value area is located under the first parameter line and extends from the first set point to the value orb. A numerical parameter value is located in the parameter area. The first parameter line represents a first range of travel of the foot pedal, and the second parameter line represents a second range of travel of the foot pedal. The first parameter line represents a first range of parameter values from the first set point to the second set point, and the second parameter line represents a second range of parameter values from the second set point to the third set point. The GUI may also comprise a fourth set point, a transition line, and a third parameter line extending from the transition line to the fourth set point.

In another example, A method for configuring a surgical system comprises: providing a GUI wherein the GUI comprises a set point icon, the set point icon comprising a first set point display and associated first set point orb, a second set point display and associated second set point orb, a first parameter line terminating at the second set point orb, and a second parameter line extending from the first set point orb; and configuring a parameter of the surgical system by movement of the first set point orb and the second set point orb. Movement of the first set point orb results in a change in a first value in the first set point display and movement of the second set pint orb results in a change in a second value in the second set point display. In addition the GUI may include a third set point display and associated third set point orb, and a third parameter line, the third parameter line extending from the second set point or, the second parameter line terminating at the second set point orb. In such a case, configuring a parameter of the surgical system further comprises configuring the parameter by movement of the third set point orb.

In another example, a computer readable medium has a set of computer instructions for providing a GUI wherein the GUI comprises a set point icon, the set point icon comprising a first set point display and associated first set point orb, a second set point display and associated second set point orb, a first parameter line terminating at the second set point orb, and a second parameter line extending from the first set point orb; and configuring a parameter of the surgical system by movement of the first set point orb and the second set point orb. Movement of the first set point orb results in a change in a first value in the first set point display and movement of the second set pint orb results in a change in a second value in the second set point display. In addition the GUI may include a third set point display and associated third set point orb, and a third parameter line, the third parameter line extending from the second set point or, the second parameter line terminating at the second set point orb. In such a case, configuring a parameter of the surgical system further comprises configuring the parameter by movement of the third set point orb.

It is to be understood that both the foregoing general description and the following detailed description are exemplary and explanatory only and are intended to provide further explanation of the invention as claimed. The following description, as well as the practice of the invention, set forth and suggest additional advantages and purposes of the invention.

### BRIEF DESCRIPTION OF THE FIGURES

The accompanying drawings, which are incorporated in and constitute a part of this specification, illustrate several embodiments of the invention and together with the description, serve to explain the principles of the invention.

FIG. 1 is a diagrammatic representation of one embodiment of a surgical console.

FIG. 2 is a representation of one embodiment of a graphical user interface (GUI).

FIG. 3 is a representation of one embodiment of a graphical user interface (GUI).

FIG. 4 is a representation of one embodiment of a graphical user interface (GUI).

FIG. 5 is a representation of one embodiment of a graphical user interface (GUI).

FIG. 6 is a representation of one embodiment of a graphical user interface (GUI).

FIG. 7 is a representation of one embodiment of a graphical user interface (GUI).

FIG. 8 is a representation of one embodiment of a graphical user interface (GUI).

FIG. 9 is a representation of one embodiment of a graphical user interface (GUI).

FIG. 10 is a representation of one embodiment of a graphical user interface (GUI).

FIG. 11 is a representation of one embodiment of a graphical user interface (GUI).

FIG. 12 is a representation of one embodiment of a graphical user interface (GUI).

FIG. 13 is a representation of one embodiment of a graphical user interface (GUI).

### DETAILED DESCRIPTION

Reference is now made in detail to the exemplary embodiments of the invention, examples of which are illustrated in the accompanying drawings. Wherever possible, the same reference numbers are used throughout the drawings to refer to the same or like parts.

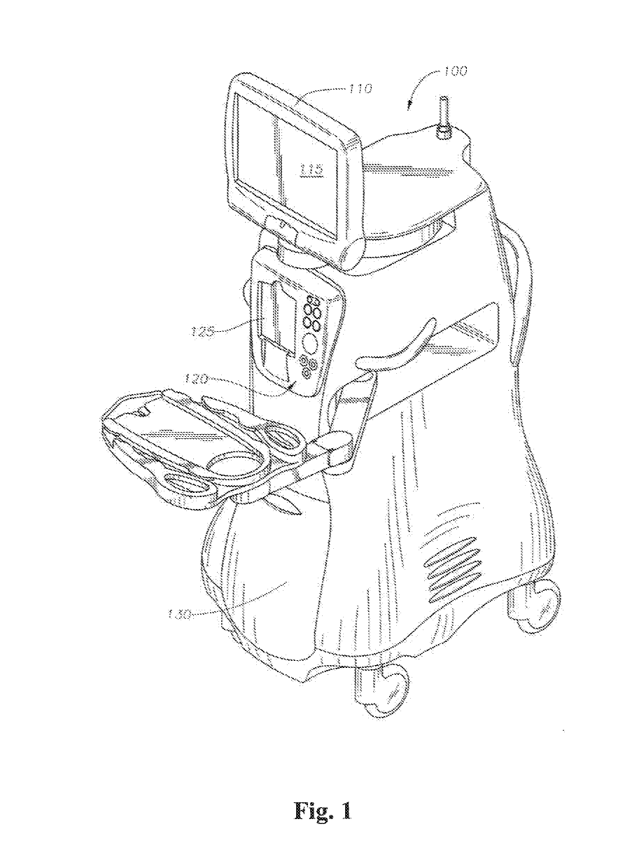

FIG. 1 is a representation of an ophthalmic surgical console 100. Surgical console 100 can include a swivel monitor 110 that has touch screen 115. Swivel monitor 110 can be positioned in a variety of orientations for whomever needs to see touch screen 115. Swivel monitor 110 can swing from side to side, as well as rotate and tilt. Touch screen 115 provides a GUI that allows a user to interact with console 100.

Surgical console 100 also includes a connection panel 120 used to connect various tools and consumables to surgical console 100. Connection panel 120 can include, for example, connectors for various hand pieces and a fluid management system (“FMS”) or cassette receiver 125. Surgical console 100 can also include a variety of features, such as a foot pedal control (e.g., stored behind panel 130) and other features.

Surgical console 100 is provided by way of example and embodiments of the present invention can be implemented with a variety of surgical systems. For example surgical console 100 may be a phacoemulsification system, vitreoretinal system, or other ophthalmic system. Embodiments of the present invention can be implemented in other suitable surgical systems having a touch screen as would be understood by one of ordinary skill in the art.

In operation, a GUI may be displayed on screen 115, such that a user may interact with the surgical console 100. In one embodiment, the GUI for a surgical system may allow a user (e.g. doctor, surgeon, or nurse) to program settings for a procedure performed by surgical console 100. In such a case, the user may be able to configure parameters or modes for the desired functionality and utilize this functionality. In other embodiments, the GUI may present data to the user so that the user can readily see how the surgical console is operating. For example, data about the state of the surgical procedure or a parameter associated with the surgical procedure can be displayed.

In general, embodiments of the present invention present parametric data in a readily viewable format to the user. Various console set points and parameters can be displayed both in numerical and graphical format on the same GUI icon. In such a manner, a wide range of information can be displayed in a compact format on the touch screen 115. In other embodiments of the present invention, operating parameters can be set by the user with a simple GUI. These set points can then be displayed on a GUI icon.

FIGS. 2-4 are examples of a GUI that may be displayed on touch screen 115. In FIGS. 2-4, a representation of aspiration flow is displayed. The aspiration flow (or flow of fluid and lens particles) out of the eye is one of many surgical parameters that is monitored by the surgeon during cataract surgery. FIGS. 2-4 depict an example of a GUI that displays aspiration flow in a simple manner. The GUI of FIGS. 2-4 include both numerical and graphical information related to aspiration flow. As explained herein, a user can readily see the following basic information related to aspiration flow: current aspiration flow value, the relative value of aspiration flow along a continuum of possible aspiration flow values; the various set points for aspiration flow; and the foot switch position for the ranges of aspiration flow. While the examples of FIGS. 2-4 are for aspiration flow, any of a number of different parameters may be displayed in a similar manner.

In FIG. 2, the value for aspiration flow is displayed in cubic centimeters per minute (as shown in the title). The parameter icon 205 of FIG. 2 displays aspiration flow for the idle state of the console (or when the foot pedal is not depressed by the user). In the example of FIG. 2, parameter icon 205 includes a parameter area 210 that displays the parameter information, a first parameter line 220, a second parameter line 230, a first set point 240, a second set point 250, a third set point 260, a parameter numerical value 270, and a mid-line 280. In this case, the parameter area 210 displays information related to aspiration flow including the following: a numerical value for aspiration flow in real time as indicated by parameter numerical value 270; three set points for aspiration flow as indicated by first, second and third set points 9240, 250, and 260, respectively); and two continuous lines that graphically display a continuum of aspiration flow values as indicated by first and second parameter lines (220 and 230, respectively).

In FIG. 2, the first and second parameter lines display a continuum of possible parameter values for a given foot switch position. In general, a foot switch is used to control operation of surgical console 100. The foot switch typically includes a pedal (like a gas pedal on a car) and additional side switches—all of which can be operated by a user's foot. The position of the users foot on the foot switch (or the amount of pedal displacement) controls the operation of the console and various parameters. For example, if the foot pedal of the foot switch is depressed a certain amount, a control signal is sent to the surgical console 100 to command a certain aspiration flow rate. In this manner, the relative position of the foot pedal on the foot switch corresponds to a particular parameter value. In the example of FIG. 2, the idle state is shown. In the idle state, the foot pedal is not depressed and there is no aspiration flow. As the foot pedal of the foot switch is depressed, the aspiration flow commanded by the console 100 varies. In this case, the first parameter line shows that the aspiration flow varies from a first set point of zero to a second set point of 30. As the foot pedal of the foot switch is depressed in a range called foot pedal position 2 (or FP2), aspiration flow can be controlled so that is gradually increases from a value of zero (indicated by first set point 240) to a value of 30 (indicated by second set point 250). Mid-line 280 visually indicates when the foot pedal position transitions from foot pedal position 2 (FP2) to foot pedal position 3 (FP3). As the foot pedal is depressed in FP3, aspiration flow gradually decreases from a value of 30 (as indicated by second set point 250) to a value of 25 (as indicated by third set point 260). In this manner, the relationship between foot pedal position and a given parameter is displayed both numerically and graphically. This allows a user to readily see foot pedal position and a corresponding parameter value in a simple parameter icon 205.

FP2 and FP3 represent ranges of travel of a given foot pedal on a foot switch. For example, FP2 may include a range of foot pedal travel from zero (foot pedal not depressed at all) to 50% (foot pedal depressed half way or 50%). In such a case, FP3 may include a range of foot pedal travel from 50% to 100% (or from foot pedal depressed half way to foot pedal fully depressed).

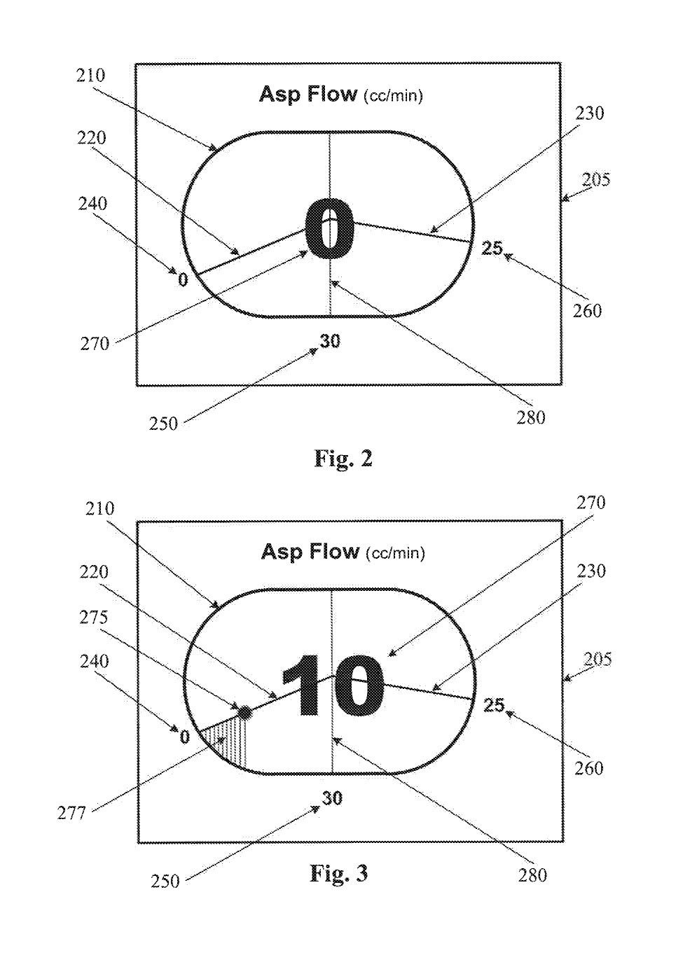

FIG. 3 shows a parameter value when the foot pedal of the foot switch is depressed in FP2. In the example of FIG. 3, parameter icon 205 includes a parameter area 210 that displays the parameter information, a first parameter line 220, a second parameter line 230, a first set point 240, a second set point 250, a third set point 260, a parameter numerical value 270, a value orb 275, a value area display 277, and a mid-line 280. The value orb 275 is located at a point along first parameter line 220 corresponding to the current foot pedal position. In this case, value orb 275 is located on first parameter line 220 about one third of the way between the first set point 540 and second set point 250. This location of value orb 275 corresponds to depressing the foot pedal about one third of the way in FP2. In other words, the location of value orb 275 along first parameter line 220 is proportional to the displacement of the foot pedal in FP2—because in this case, first parameter line 220 corresponds to the full length of foot pedal travel in FP2. In addition to value orb 275, parameter value display 277 graphically shows the distance along first parameter line 220 that value orb 275 travels. Value parameter display 277 provides a shading or colored area under first parameter line 220 that corresponds to the location of value orb 275 on first parameter line 220 (and which also corresponds to the foot pedal position in FP2). The parameter numerical value 270 is displayed as a large number in the middle of parameter area 210. In this case, aspiration flow is ten cc/min. Parameter numerical value 270 corresponds to the position of value orb 275 on first parameter line 220 which also corresponds to the amount of shaded area shown by parameter value display 277 which in turn corresponds to the foot pedal position in FP2. As the foot pedal is further depressed in FP2, value orb 275 travels to the right along first parameter line 220, the corresponding parameter value display 277 increases in area, and the parameter numerical value 270 increases.

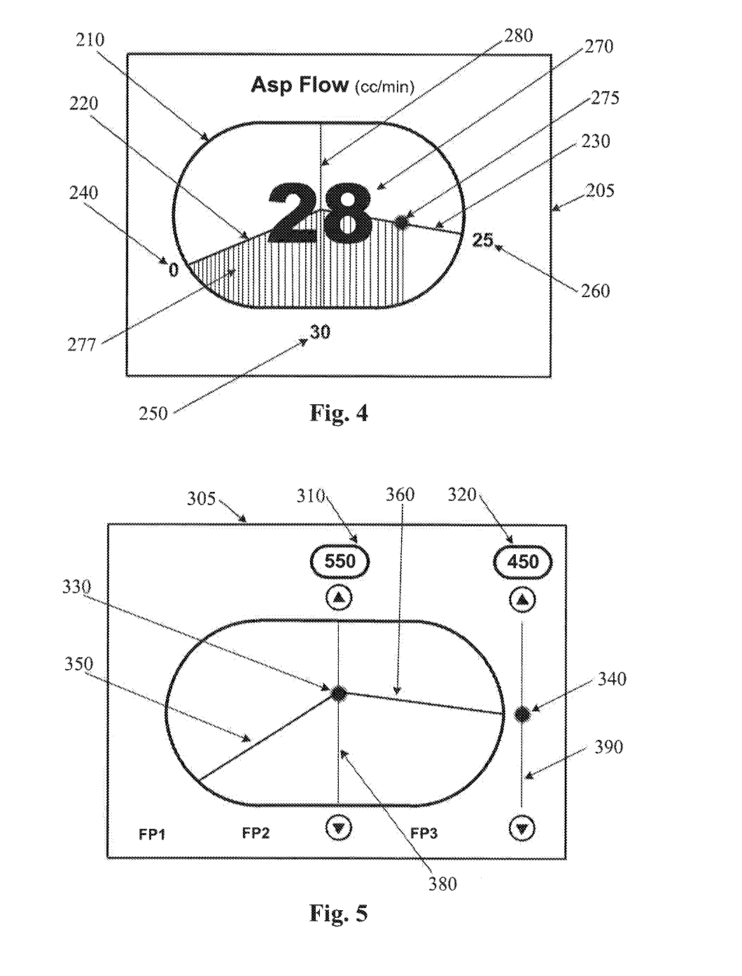

When the value orb 275 reaches the mid-line 280, the value orb 275 can continue to travel to right as seen in FIG. 4. The mid-line 280 indicates the boundary between FP2 and FP3. Typically, surgical console 100 and its accompanying foot switch are programmable such that the various ranges of travel of the foot pedal on the foot switch can be programmed as FP2 and FP3. If the transition between FP2 and FP3 is indicated by depressing the foot pedal 50% or half way, then mid-line 280 provides a visual indication of this transition point. In other words, as the foot pedal is depressed from zero to 50%, value orb 275 travels along first parameter line 220 from the first set point 240 to the mid-line 280 (in FP2). As the foot pedal is depressed from 50% to 100%, value orb 275 travels along second parameter line 230 from the mid-line 280 to the third set point 260 (in FP3).

As seen in the example of FIG. 4, value orb 275 is located along second parameter line 230 about mid-way between mid-line 280 and third set point 260. This location of value orb 275 corresponds to a foot pedal position about half way through FP3. In addition, value area display 277 shows the total foot pedal displacement graphically. Value area display 277 provides a shaded or colored area beneath both first parameter line 220 and second parameter line 230 that indicates the total travel of value orb 275 (and related total travel of the foot pedal).

While the parameter area 210 is shown as an obround or pill shape, any of a number of other different shapes may be employed. Value orb 275 may also be represented by different graphics as may value display area 277. While the relationship between foot pedal position, value orb 275, value display area 277, and parameter value 270 is described, a similar relationship can exist between any other control device. In the example above the control device is the foot switch (and its foot pedal). Other types of control devices include, but are not limited to the following: switches, dials, hand held controls, foot operated controls, pedals, and the like. In this manner, the relative position of control device corresponds to a value orb 275, value display area 277, and parameter value 270 as displayed in a parameter icon 210.

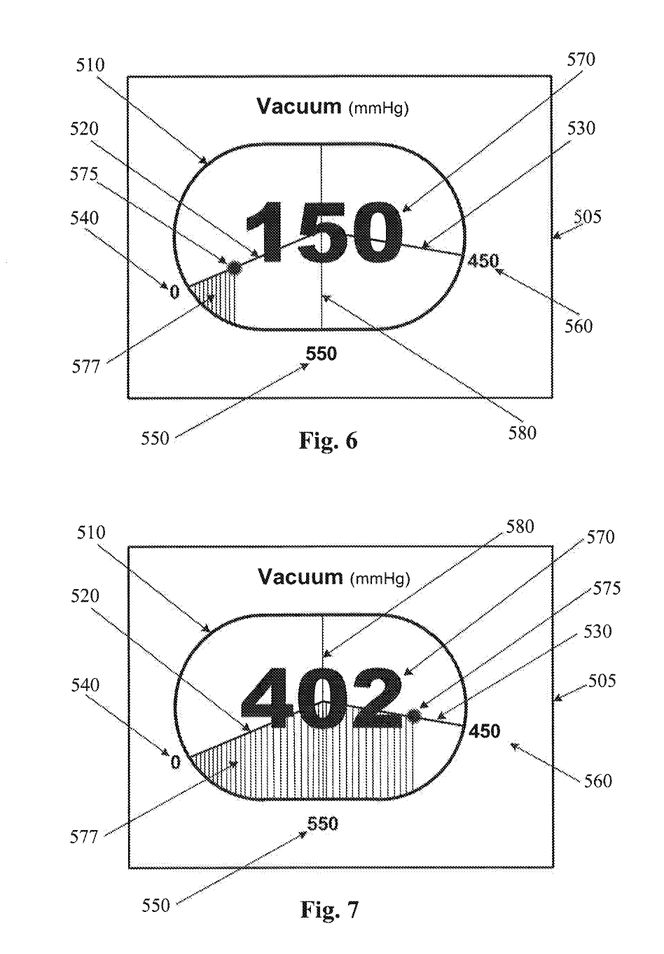

FIG. 5 is an example of a GUI that can be used to change set points for a parameter icon. The GUI of FIG. 5 is used to change the set points for the parameter icon of FIGS. 6 and 7. In this case, the GUI of FIG. 5 provides an intuitive way to change set points for vacuum. Vacuum level is one parameter that is typically set by the surgeon for a cataract procedure. During surgery, the vacuum level is controlled by the foot switch position.

The GUI of FIG. 5 includes a set point icon 305 that has a second set point display 210 (that corresponds second set point 550 of FIGS. 5 and 6) and a third set point display 320 (that corresponds to third set point 560 of FIGS. 5 and 6). Set point icon 305 also includes a second set point orb 330; a third set point orb 340; a second set point line 380; a third set point line 390; a first parameter line 350; and a second parameter line 360. In general, the arrows located above and below second set point line 380 can be used to move second set point orb 330 up and down along second set point line 380 thus changing the set point value that is displayed in second set point display 310. Likewise, the arrows located above and below third set point line 390 can be used to move third set point orb 340 up and down along second set point line 390 thus changing the set point value that is displayed in third set point display 320. When the GUI of FIG. 5 is displayed on touch screen 115, a user can simply touch the second or third set point orb (330 and 340 respectively) and drag the orb to a desired location. In other words, the set point orbs (330 and 340) can be moved by dragging one's finger across the touch screen 115 or by using the arrows located at each end of the set point lines (380 and 390). Second set point orb 330 is coupled to one end of first parameter line 350 and one end of second parameter line 360. As second set point orb 330 moves up and down along second set point line 380, the ends of first and second parameter lines (350 and 360 respectively) also move up and down. In this manner, second set point orb 330 is fixed to one end of first parameter line 350 and one end of second parameter line 360. Likewise, moving third set point orb 340 up and down results in moving the other end of second parameter line 360 up and down. In this manner, the relative slopes of first and second parameter lines (350 and 360) are also adjusted by movement of second and third set point orbs (330 and 340). As the second and third set point orbs 330 and 340 are moved, the value displayed in second and third set point displays 310 and 320 also change. Alternatively, a user may enter a numerical value in second and third set point displays 310 and 320 with a key board or the like.

FIGS. 6 and 7 are examples of parameter icons whose set points have been changed by the set point icon 305. FIGS. 6 and 7 are examples of a GUI that may be displayed on touch screen 115. In FIGS. 6 and 7, a representation of vacuum is displayed. The vacuum (or vacuum level) is one of many surgical parameters that is set by the surgeon for cataract surgery. FIGS. 6 and 7 depict an example of a GUI that displays vacuum in a simple manner. The GUI of FIGS. 6 and 7 include both numerical and graphical information related to aspiration flow. As explained herein, a user can readily see the following basic information related to vacuum: current vacuum value, the relative value of vacuum along a continuum of possible vacuum values; the various set points for vacuum; and the foot switch position for the ranges of vacuum. While the examples of FIGS. 6 and 7 are vacuum, any of a number of different parameters vacuum may be displayed in a similar manner.

In FIG. 6, the first and second parameter lines display a continuum of possible parameter values for a given foot switch position. In general, a foot switch is used to control operation of surgical console 100. The foot switch typically includes a pedal (like a gas pedal on a car) and additional side switches—all of which can be operated by a user's foot. The position of the users foot on the foot switch (or the amount of pedal displacement) controls the operation of the console and various parameters. For example, if the foot pedal of the foot switch is depressed a certain amount, a control signal is sent to the surgical console 100 to command a certain aspiration flow rate. In this manner, the relative position of the foot pedal on the foot switch corresponds to a particular parameter value. In the example of FIG. 6, as the foot pedal of the foot switch is depressed, the vacuum commanded by the console 100 varies. In this case, the first parameter line shows that the vacuum varies from a first set point of zero to a second set point of 550. As the foot pedal of the foot switch is depressed in a range called foot pedal position 2 (or FP2), vacuum can be controlled so that is gradually increases from a value of zero (indicated by first set point 540) to a value of 550 (indicated by second set point 550). Mid-line 580 visually indicates when the foot pedal position transitions from foot pedal position 2 (FP2) to foot pedal position 3 (FP3). As the foot pedal is depressed in FP3, vacuum gradually decreases from a value of 550 (as indicated by second set point 550) to a value of 450 (as indicated by third set point 560). In this manner, the relationship between foot pedal position and a given parameter is displayed both numerically and graphically. This allows a user to readily see foot pedal position and a corresponding parameter value in a simple parameter icon 505.

FIG. 6 shows a parameter value when the foot pedal of the foot switch is depressed in FP2. In the example of FIG. 6, parameter icon 505 includes a parameter area 510 that displays the parameter information, a first parameter line 520, a second parameter line 530, a first set point 540, a second set point 550, a third set point 560, a parameter numerical value 570, a value orb 575, a value area display 577, and a mid-line 580. The value orb 575 is located at a point along first parameter line 520 corresponding to the current foot pedal position. In this case, value orb 575 is located on first parameter line 520 about one third of the way between the first set point 540 and second set point 550. This location of value orb 575 corresponds to depressing the foot pedal about one third of the way in FP2. In other words, the location of value orb 575 along first parameter line 520 is proportional to the displacement of the foot pedal in FP2—because in this case, first parameter line 520 corresponds to the full length of foot pedal travel in FP2. In addition to value orb 575, parameter value display 577 graphically shows the distance along first parameter line 520 that value orb 575 travels. Value parameter display 577 provides a shading or colored area under first parameter line 520 that corresponds to the location of value orb 575 on first parameter line 520 (and which also corresponds to the foot pedal position in FP2). The parameter numerical value 570 is displayed as a large number in the middle of parameter area 510. In this case, vacuum is 150 mmHg. Parameter numerical value 570 corresponds to the position of value orb 575 on first parameter line 520 which also corresponds to the amount of shaded area shown by parameter value display 577 which in turn corresponds to the foot pedal position in FP2. As the foot pedal is further depressed in FP2, value orb 575 travels to the right along first parameter line 520, the corresponding parameter value display 577 increases in area, and the parameter numerical value 570 increases.

When the value orb 575 reaches the mid-line 580, the value orb 575 can continue to travel to right as seen in FIG. 7. The mid-line 580 indicates the boundary between FP2 and FP3. Typically, surgical console 100 and its accompanying foot switch are programmable such that the various ranges of travel of the foot pedal on the foot switch can be programmed as FP2 and FP3. If the transition between FP2 and FP3 is indicated by depressing the foot pedal 50% or half way, then mid-line 580 provides a visual indication of this transition point. In other words, as the foot pedal is depressed from zero to 50%, value orb 575 travels along first parameter line 520 from the first set point 540 to the mid-line 580 (in FP2). As the foot pedal is depressed from 50% to 100%, value orb 575 travels along second parameter line 530 from the mid-line 580 to the third set point 560 (in FP3).

As seen in the example of FIG. 7, value orb 575 is located along second parameter line 530 about mid-way between mid-line 580 and third set point 560. This location of value orb 575 corresponds to a foot pedal position about half way through FP3. In addition, value area display 577 shows the total foot pedal displacement graphically. Value area display 577 provides a shaded or colored area beneath both first parameter line 520 and second parameter line 530 that indicates the total travel of value orb 575 (and related total travel of the foot pedal).

While the parameter area 510 is shown as an obround or pill shape, any of a number of other different shapes may be employed. Value orb 575 may also be represented by different graphics as may value display area 577. While the relationship between foot pedal position, value orb 575, value display area 577, and parameter value 570 is described, a similar relationship can exist between any other control device. In the example above the control device is the foot switch (and its foot pedal). Other types of control devices include, but are not limited to the following: switches, dials, hand held controls, foot operated controls, pedals, and the like. In this manner, the relative position of control device corresponds to a value orb 575, value display area 577, and parameter value 570 as displayed in a parameter icon 510.

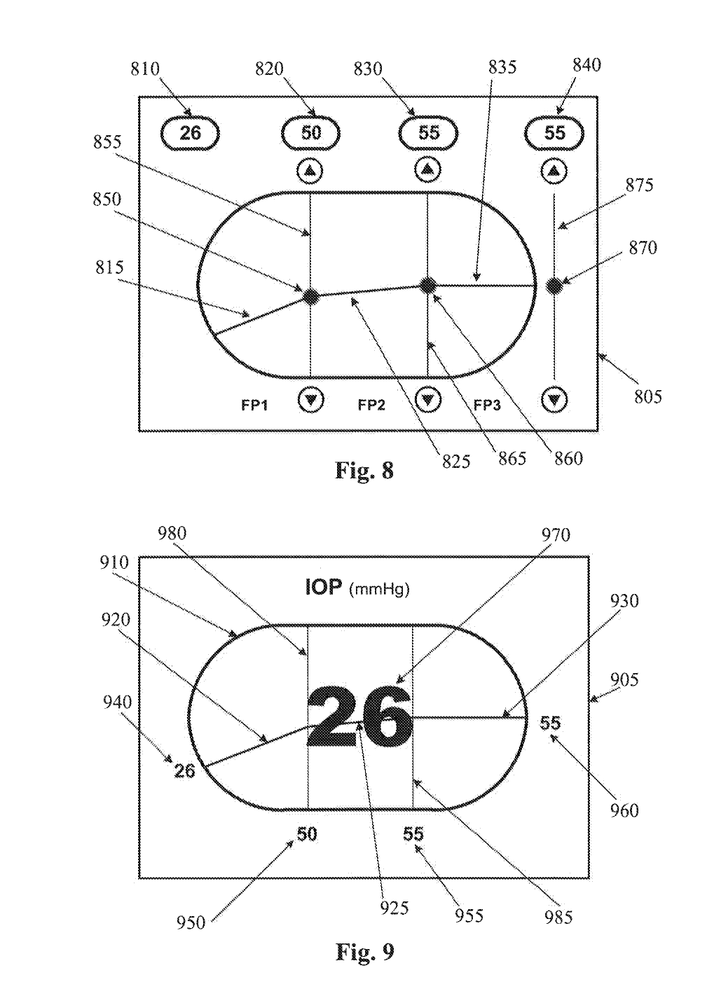

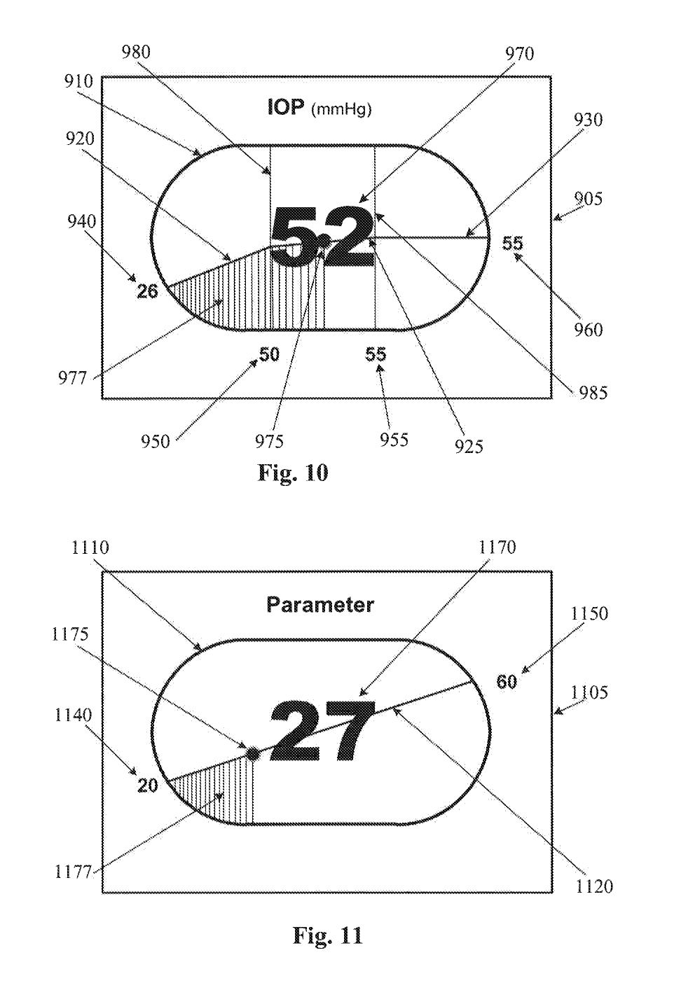

FIG. 8 is an example of a GUI that can be used to change set points for a parameter icon. The GUI of FIG. 8 is used to change the set points for the parameter icon of FIGS. 9-10. In this case, the GUI of FIG. 8 provides an intuitive way to change set points for intraocular pressure (IOP). IOP is one parameter that is typically set by the surgeon for a cataract procedure. During surgery, the IOP level may be controlled by the foot switch position.

The GUI of FIG. 8 includes a set point icon 805 that has a first set point display 810 (that corresponds to first set point 940 of FIGS. 9 and 10), a second set point display 820 (that corresponds to second set point 950 of FIGS. 9 and 10), a third set point display 830 (that corresponds to third set point 955 of FIGS. 9 and 10), and a fourth set point display 840 (that corresponds to fourth set point 960 of FIGS. 9 and 10). Set point icon 805 also includes a second set point orb 850, a third set point orb 860, a fourth set point orb 870, a second set point line 855; a third set point line 865, a fourth set point line 875, a first parameter line 815, a second parameter line 825, and a third parameter line 835.

In general, the arrows located above and below second set point line 855 can be used to move second set point orb 850 up and down along second set point line 855 thus changing the set point value that is displayed in second set point display 820. Likewise, the arrows located above and below third set point line 865 can be used to move third set point orb 860 up and down along second set point line 865 thus changing the set point value that is displayed in third set point display 830. The arrows located above and below third set point line 875 can be used to move third set point orb 870 up and down along second set point line 875 thus changing the set point value that is displayed in third set point display 840. When the GUI of FIG. 8 is displayed on touch screen 115, a user can simply touch the second, third or fourth set point orb (850, 860, and 870 respectively) and drag the orb to a desired location. In other words, the set point orbs (850, 860, and 870) can be moved by dragging one's finger across the touch screen 115 or by using the arrows located at each end of the set point lines (855, 865, and 875). Second set point orb 850 is coupled to one end of first parameter line 815 and one end of second parameter line 825. As second set point orb 850 moves up and down along second set point line 855, the ends of first and second parameter lines (815 and 825 respectively) also move up and down. In this manner, second set point orb 850 is fixed to one end of first parameter line 815 and one end of second parameter line 825. Likewise, moving third set point orb 860 up and down results in moving the other end of second parameter line 825 up and down. In this manner, the relative slopes of first, second, and third parameter lines (815, 825, and 835) are also adjusted by movement of second, third, and fourth set point orbs (850, 860, and 870). As second, third, and fourth set point orbs (850, 860, and 870) are moved, the value displayed in second, third, and fourth set point displays 820, 830, and 840 also change. Alternatively, a user may enter a numerical value in any of the set point displays 810, 820, 830, and 840 with a key board or the like.

FIGS. 9 and 10 are examples of parameter icons whose set points have been changed by the set point icon 805. FIGS. 9 and 10 are examples of a GUI that may be displayed on touch screen 115. In FIGS. 9 and 10, a representation of IOP is displayed. The IOP (or IOP level) is one of many surgical parameters that is set by the surgeon for cataract surgery. FIGS. 9 and 10 depict an example of a GUI that displays IOP in a simple manner. The GUI of FIGS. 9 and 10 include both numerical and graphical information related to aspiration flow. As explained herein, a user can readily see the following basic information related to IOP: current IOP value, the relative value of IOP along a continuum of possible IOP values; the various set points for IOP; and the foot switch position for the ranges of IOP. While the examples of FIGS. 9 and 10 are IOP, any of a number of different parameters IOP may be displayed in a similar manner.

In FIG. 9, the value for IOP is displayed in millimeters of mercury (as shown in the title). The parameter icon 905 of FIG. 2 displays IOP for the idle state of the console (or when the foot pedal is not depressed by the user). In the example of FIG. 9, parameter icon 905 includes a parameter area 910 that displays the parameter information, a first parameter line 920, a second parameter line 925, a third parameter line 930, a first set point 940, a second set point 950, a third set point 955, a fourth set point 960, a parameter numerical value 970, and transition lines 980 and 985. In this case, the parameter area 910 displays information related to IOP including the following: a numerical value for IOP in real time as indicated by parameter numerical value 970; four set points for IOP as indicated by first, second, third, and fourth set points (940, 950, 955, and 960, respectively); and three continuous lines that graphically display a continuum of IOP values as indicated by first, second, and third parameter lines (920, 925, and 930, respectively).

In FIGS. 9 and 10, the first second, and third parameter lines display a continuum of possible parameter values for a given foot switch position. In general, a foot switch is used to control operation of surgical console 100. The foot switch typically includes a pedal (like a gas pedal on a car) and additional side switches—all of which can be operated by a user's foot. The position of the user's foot on the foot switch (or the amount of pedal displacement) controls the operation of the console and various parameters. For example, if the foot pedal of the foot switch is depressed a certain amount, a control signal is sent to the surgical console 100 to control a certain IOP. In this manner, the relative position of the foot pedal on the foot switch corresponds to a particular parameter value. In the example of FIGS. 9 and 10, as the foot pedal of the foot switch is depressed, the IOP controlled by the console 100 varies. In this case, the first parameter line shows that the IOP varies from a first set point of 26 to a second set point of 50. As the foot pedal of the foot switch is depressed in a range called foot pedal position 1 (or FP1), IOP can be controlled so that is gradually increases from a value of 26 (indicated by first set point 940) to a value of 50 (indicated by second set point 950). Transition line 580 visually indicates when the foot pedal position transitions from foot pedal position 1 (FP1) to foot pedal position 2 (FP2). As the foot pedal is depressed in FP2, IOP gradually increases from a value of 50 (as indicated by second set point 950) to a value of 55 (as indicated by third set point 955). Transition line 985 visually indicates when the foot pedal position transitions from foot pedal position 2 (FP2) to foot pedal position 3 (FP3). As the foot pedal is depressed in FP3, IOP gradually remains at 55 (as indicated by second set point 950 and third set point 955). In this manner, the relationship between foot pedal position and a given parameter is displayed both numerically and graphically. This allows a user to readily see foot pedal position and a corresponding parameter value in a simple parameter icon 905. Notice also that the FP1, FP2, and FP3 are also shown in FIG. 8 and have the same meaning in that Figure.

FIG. 10 shows a parameter value when the foot pedal of the foot switch is depressed in FP2. In the example of FIG. 10, parameter icon 905 includes a parameter area 910 that displays the parameter information, a first parameter line 920, a second parameter line 925, a third parameter line 930, a first set point 940, a second set point 950, a third set point 955, a fourth set point 960, a parameter numerical value 970, value orb 975, parameter value display 977, and transition lines 980 and 985. The value orb 975 is located at a point along second parameter line 925 corresponding to the current foot pedal position. In this case, value orb 975 is located on second parameter line 925 about half of the way between the second set point 950 and third set point 955. This location of value orb 975 corresponds to depressing the foot pedal about one half of the way in FP2. In other words, the location of value orb 975 along second parameter line 925 is proportional to the displacement of the foot pedal in FP2—because in this case, second parameter line 925 corresponds to the full length of foot pedal travel in FP2. In addition to value orb 975, parameter value display 977 graphically shows the distance along first parameter line 920 and second parameter line 925 that value orb 975 traveled. Value parameter display 977 provides a shading or colored area under first and second parameter lines 920 and 925 that corresponds to the location of value orb 975 on second parameter line 925 (and which also corresponds to the foot pedal position in FP2). The parameter numerical value 970 is displayed as a large number in the middle of parameter area 510. In this case, IOP is 52 mmHg. Parameter numerical value 970 corresponds to the position of value orb 975 on second parameter line 925 which also corresponds to the amount of shaded area shown by parameter value display 977 which in turn corresponds to the foot pedal position in FP2. As the foot pedal is further depressed in FP2, value orb 975 travels to the right along second parameter line 925, the corresponding parameter value display 977 increases in area, and the parameter numerical value 970 increases.

When the value orb 975 reaches transition line 985, the value orb 975 can continue to travel to right. The transition line 985 indicates the boundary between FP2 and FP3. Typically, surgical console 100 and its accompanying foot switch are programmable such that the various ranges of travel of the foot pedal on the foot switch can be programmed as FP2 and FP3. If the transition between FP2 and FP3 is indicated by depressing the foot pedal 70%, then mid-line 580 provides a visual indication of this transition point. In other words, as the foot pedal is depressed from zero to 70%, value orb 575 travels along first and second parameter lines 920 and 925 from the first set point 940 to the second set point 950 and to the transition line 585. As the foot pedal is depressed from 70% to 100%, value orb 975 travels along third parameter line 930 from the transition line 585 to the third set point 960 (in FP3).

While the parameter area 510 is shown as an obround or pill shape, any of a number of other different shapes may be employed. Value orb 575 may also be represented by different graphics as may value display area 577. While the relationship between foot pedal position, value orb 575, value display area 577, and parameter value 570 is described, a similar relationship can exist between any other control device. In the example above the control device is the foot switch (and its foot pedal). Other types of control devices include, but are not limited to the following: switches, dials, hand held controls, foot operated controls, pedals, and the like. In this manner, the relative position of control device corresponds to a value orb 575, value display area 577, and parameter value 570 as displayed in a parameter icon 510.

FIG. 11 is an example of a GUI that may be displayed on touch screen 115. In FIG. 11, a representation of a parameter is displayed. The GUI of FIG. 11 includes both numerical and graphical information related to the parameter. As explained herein, a user can readily see the following basic information related to the parameter: current parameter value, the relative value of the parameter along a continuum of possible the parameter values; the various set points for the parameter; and the foot switch position for the ranges of the parameter.

In FIG. 11, the parameter icon 1105 displays includes a parameter area 1110 that displays the parameter information, a parameter line 1120, a first set point 1140, a second set point 1150, and a parameter numerical value 1170. In this case, the parameter area 1110 displays information related to the parameter including the following: a numerical value for parameter in real time as indicated by parameter numerical value 1170; two set points for the parameter as indicated by first and second set points 1140 and 1150; and one continuous line that graphically displays a continuum of parameter values as indicated by parameter line 1120.

In FIG. 11, the parameter line 1120 displays a continuum of possible parameter values for a given foot switch position. In general, a foot switch is used to control operation of surgical console 100. The foot switch typically includes a pedal (like a gas pedal on a car) and additional side switches—all of which can be operated by a user's foot. The position of the user's foot on the foot switch (or the amount of pedal displacement) controls the operation of the console and various parameters. For example, if the foot pedal of the foot switch is depressed a certain amount, a control signal is sent to the surgical console 100 to control or affect a certain. In this manner, the relative position of the foot pedal on the foot switch corresponds to a particular parameter value. In the example of FIG. 11, as the foot pedal of the foot switch is depressed, the parameter value controlled or affected by the console 100 varies. In this case, the parameter line 1120 shows that the parameter varies from a first set point of 20 to a second set point of 60. As the foot pedal of the foot switch is depressed, the parameter can be controlled or affected so that is gradually increases from a value of 20 (indicated by first set point 1140) to a value of 60 (indicated by second set point 1150). In addition, the current parameter value (27) is displayed as parameter numerical value 1170. In this manner, the relationship between foot pedal position and a given parameter is displayed both numerically and graphically. This allows a user to readily see foot pedal position and a corresponding parameter value in a simple parameter icon 205.

FIG. 11 shows a parameter value when the foot pedal of the foot switch is depressed. In the example of FIG. 11, parameter icon 1105 includes a parameter area 1110 that displays the parameter information, a parameter line 1120, a first set point 1140, a second set point 1150, a value orb 1175, a parameter area display 1177, and a parameter numerical value 1170. The value orb 1175 is located at a point along parameter line 1120 corresponding to the current foot pedal position. In this case, value orb 1175 is located on parameter line 1120 about 25% of the way between the first set point 1140 and second set point 1150. This location of value orb 1175 corresponds to depressing the foot pedal about 25%. In other words, the location of value orb 1175 along parameter line 1120 is proportional to the displacement of the foot pedal—because in this case, parameter line 1120 corresponds to the full length of foot pedal travel. In addition to value orb 1175, parameter value display 1177 graphically shows the distance along first parameter line 1120 that value orb 1175 travels. Value parameter display 1177 provides a shading or colored area under parameter line 1120 that corresponds to the location of value orb 1175 on parameter line 1120 (and which also corresponds to the foot pedal position). The parameter numerical value 1170 is displayed as a large number in the middle of parameter area 1110. Parameter numerical value 1170 corresponds to the position of value orb 1175 on parameter line 1120 which also corresponds to the amount of shaded area shown by parameter value display 1177 which in turn corresponds to the foot pedal position. As the foot pedal is further depressed, value orb 1175 travels to the right along parameter line 1120, the corresponding parameter value display 1177 increases in area, and the parameter numerical value 1170 increases.

While the parameter area 1110 is shown as an obround or pill shape, any of a number of other different shapes may be employed. Value orb 1175 may also be represented by different graphics as may value display area 1177. While the relationship between foot pedal position, value orb 1175, value display area 1177, and parameter value 1170 is described, a similar relationship can exist between any other control device. In the example above, the control device is the foot switch (and its foot pedal). Other types of control devices include, but are not limited to the following: switches, dials, hand held controls, foot operated controls, pedals, and the like. In this manner, the relative position of control device corresponds to a value orb 1175, value display area 1177, and parameter value 1170 as displayed in a parameter icon 210.

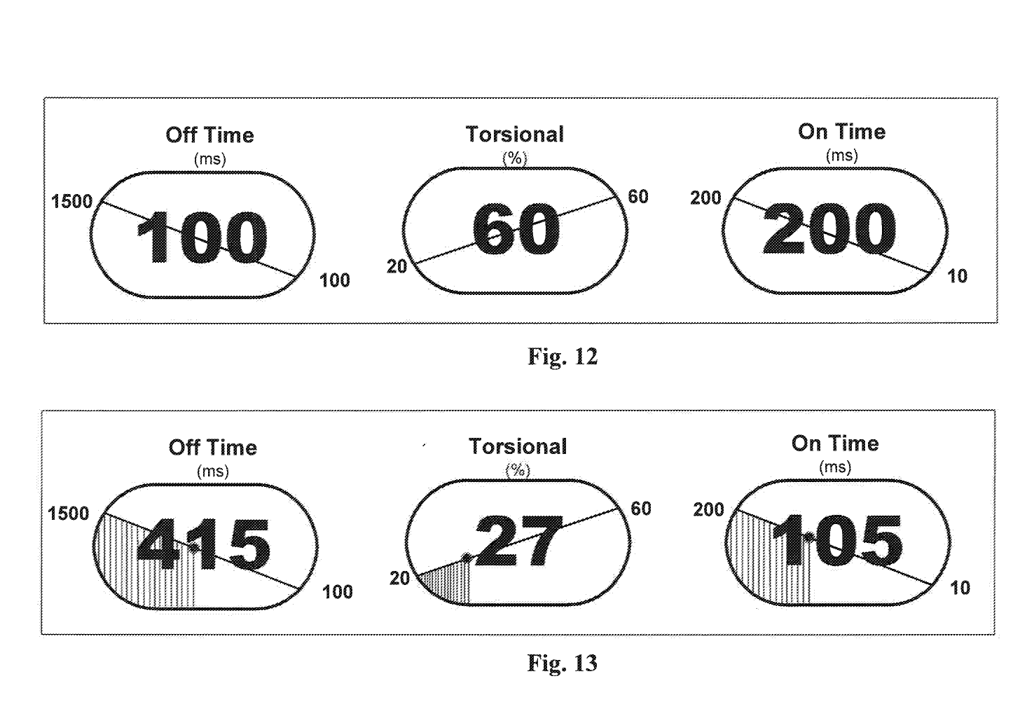

FIGS. 12 and 13 show a set of parameter icons similar to that described in FIG. 11. In FIGS. 12 and 13, a set of parameter icons displays parameters associated with ultrasonic power delivered to a surgical hand piece. Thus, FIGS. 12 and 13 demonstrate that more than one parameter icon can be used in a GUI to display a set of associated parameters. In the example of FIGS. 12 and 13, the parameters are the following: ultrasonic power on time; percentage of torsional power provided; and ultrasonic off time. The set points, parameter lines, parameter values, value orbs, and value area displays are as described with respect to previous figures and in particular, FIG. 11. The set points may be selected by a user and displayed as in FIG. 13. Alternatively, the value orbs shown in FIG. 13 may move as these parameter values change, for example, by movement of a foot pedal or other control device.

From the above, it may be appreciated that the present invention provides a GUI for a surgical console. The present disclosure describes a simplified GUI that allows a doctor to control settings for a surgical console. The GUI also presents data about the state of the surgical console or procedure (e.g. surgical parameters) to a doctor in a simple and logical fashion. The present invention is illustrated herein by example, and various modifications may be made by a person of ordinary skill in the art.

Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

What is claimed is:

1. A surgical system comprising:
a control device;
a touch screen display; and
a GUI displayed on the touch screen display, wherein the GUI comprises a parameter icon, the parameter icon comprising a parameter area, a first set point, a second set point, a parameter line extending from the first set point to the second set point, and a value orb located on the parameter line;
wherein a location of the value orb corresponds to a control position of the control device.

2. The system of claim 1 wherein the GUI further comprises:
a parameter value area located under the parameter line and extending from the first set point to the value orb.

3. The system of claim 1 wherein the GUI further comprises:
a numerical parameter value located in the parameter area.

4. The system of claim 1 wherein the control device comprises a foot pedal.

5. The system of claim 4 wherein the location of the value orb on the parameter line is proportional to a position of the foot pedal.

6. The system of claim 1 wherein the parameter line has a slope, and the parameter line represents a range of parameter values from the first set point to the second set point.

7. The system of claim 4 wherein the parameter line represents a range of travel of the foot pedal.

8. A surgical system comprising:
a control device;
a touch screen display; and
a GUI displayed on the touch screen display, wherein the GUI comprises a parameter icon, the parameter icon comprising a parameter area, a first set point, a second set point, a third set point, a mid-line, a first parameter line extending from the first set point to the mid-line, a second parameter line extending from the mid-line to the third set point, and a value orb located on the first or second parameter line;
wherein a location of the value orb corresponds to a control position of the control device.

9. The system of claim 8 wherein the GUI further comprises:
a parameter value area located under the first parameter line and extending from the first set point to the value orb.

10. The system of claim 8 wherein the GUI further comprises:
a numerical parameter value located in the parameter area.

11. The system of claim 8 wherein the control device comprises a foot pedal.

12. The system of claim 11 wherein the location of the value orb on the first or second parameter line is proportional to a position of the foot pedal.

13. The system of claim 11 wherein the first parameter line represents a first range of travel of the foot pedal, and the second parameter line represents a second range of travel of the foot pedal.

14. The system of claim 8 wherein the first parameter line represents a first range of parameter values from the first set point to the second set point, and the second parameter line represents a second range of parameter values from the second set point to the third set point.

15. The system of claim 8 wherein the GUI further comprises fourth set point, a transition line, and a third parameter line extending from the transition line to the fourth set point.

16. A computer readable medium comprising a set of computer instructions, translatable for:
providing a GUI, wherein the GUI comprises a parameter icon, the parameter icon comprising a parameter area, a first set point, a second set point, a parameter line extending from the first set point to the second set point, and a value orb located on the parameter line;
wherein a location of the value orb corresponds to a control position of a control device.

17. The computer readable medium of claim 16 wherein the GUI further comprises:
a parameter value area located under the parameter line and extending from the first set point to the value orb.

18. The computer readable medium of claim 16 wherein the GUI further comprises:
a numerical parameter value located in the parameter area.

19. The computer readable medium of claim 16 wherein the control device comprises a foot pedal.

20. The computer readable medium of claim 19 wherein the location of the value orb on the parameter line is proportional to a position of the foot pedal.

21. The computer readable medium of claim 16 wherein the parameter line has a slope, and the parameter line represents a range of parameter values from the first set point to the second set point.

22. The computer readable medium of claim 19 wherein the parameter line represents a range of travel of the foot pedal.

23. A computer readable medium comprising a set of computer instructions, translatable for:
providing a GUI, wherein the GUI comprises a parameter icon, the parameter icon comprising a parameter area, a first set point, a second set point, a third set point, a mid-line, a first parameter line extending from the first set point to the mid-line, a second parameter line extending from the mid-line to the third set point, and a value orb located on the first or second parameter line;
wherein a location of the value orb corresponds to a control position of a control device.

24. The computer readable medium of claim 23 wherein the GUI further comprises:
a parameter value area located under the first parameter line and extending from the first set point to the value orb.

25. The computer readable medium of claim 23 wherein the GUI further comprises:
a numerical parameter value located in the parameter area.

26. The computer readable medium of claim 23 wherein the control device comprises a foot pedal.

27. The computer readable medium of claim 26 wherein the location of the value orb on the first or second parameter line is proportional to a position of the foot pedal.

28. The computer readable medium of claim 26 wherein the first parameter line represents a first range of travel of the foot pedal, and the second parameter line represents a second range of travel of the foot pedal.

29. The computer readable medium of claim 23 wherein the first parameter line represents a first range of parameter values from the first set point to the second set point, and the second parameter line represents a second range of parameter values from the second set point to the third set point.

30. The computer readable medium of claim 23 wherein the GUI further comprises fourth set point, a transition line, and a third parameter line extending from the transition line to the fourth set point.

31. A method for configuring a surgical system, the method comprising:
providing a GUI wherein the GUI comprises a set point icon, the set point icon comprising a first set point display and associated first set point orb, a second set point display and associated second set point orb, a first parameter line terminating at the second set point orb, and a second parameter line extending from the first set point orb; and
configuring a parameter of the surgical system by movement of the first set point orb and the second set point orb.

32. The method of claim 31 wherein movement of the first set point orb results in a change in a first value in the first set point display and movement of the second set pint orb results in a change in a second value in the second set point display.

33. The method of claim 31 wherein providing a GUI further comprises providing a third set point display and associated third set point orb, and a third parameter line, the third parameter line extending from the second set point or, the second parameter line terminating at the second set point orb.

34. The method of claim 33 wherein configuring a parameter of the surgical system further comprises configuring the parameter by movement of the third set point orb.

35. A computer readable medium comprising a set of computer instructions, translatable for:
providing a GUI wherein the GUI comprises a set point icon, the set point icon comprising a first set point display and associated first set point orb, a second set point display and associated second set point orb, a first parameter line terminating at the second set point orb, and a second parameter line extending from the first set point orb;
wherein configuring a parameter of the surgical system is accomplished by movement of the first set point orb and the second set point orb.

36. The method of claim 35 wherein movement of the first set point orb results in a change in a first value in the first set point display and movement of the second set pint orb results in a change in a second value in the second set point display.

37. The method of claim 35 wherein providing a GUI further comprises providing a third set point display and associated third set point orb, and a third parameter line, the third parameter line extending from the second set point or, the second parameter line terminating at the second set point orb.

38. The method of claim 37 wherein configuring a parameter of the surgical system further comprises configuring the parameter by movement of the third set point orb.
